(19) United States
(12) Patent Application Publication
(10) Pub. No.: US 2017/0312222 A1
(43) Pub. Date: Nov. 2, 2017

(54) Taste-Masked Formulations of Raltegravir

(71) Applicant: Merck Sharp & Dohme Corp., Rahway, NJ (US)

(72) Inventors: Karen Cassidy Thompson, Lansdale, PA (US); Kimberly Nicole Kaighn, Glens Mills, PA (US); Indra Neil Mukherjee, West Chester, PA (US); Catherine Elizabeth Diimmler, Lansdale, PA (US); Hedy Teppler Weiser, Merion, PA (US); Christopher Mancinelli, Collegeville, PA (US)

(73) Assignee: Merck Sharp & Dohme Corp., Rahway, NJ (US)

(21) Appl. No.: 15/652,658

(22) Filed: Jul. 18, 2017

Related U.S. Application Data
(63) Continuation of application No. 14/113,341, filed on May 5, 2014.
(60) Provisional application No. 61/478,320, filed on Apr. 22, 2011.

(51) Int. Cl. A61K 9/16; A61K 31/513; A61K 31/41; A61K 9/50; A61K 9/20; A61K 45/06; A61K 9/00

(52) U.S. Cl. CPC A61K 9/1652; A61K 45/06; A61K 31/513; A61K 9/0056; A61K 9/5047; A61K 9/2081; A61K 31/41

(57) ABSTRACT

Disclosed are taste-masked pharmaceutical formulations of raltegravir comprising coated API granules mixed with a screened powder excipient blend in either tablet or sachet form. The core and coated granules are produced using a Wurster process for enhanced control of particle size. Also disclosed are methods of treating HIV, e.g., in pediatric populations.

### FIELD OF THE INVENTION

The present invention is directed to taste-masked granules that contain raltegravir or a pharmaceutically acceptable salt thereof, to oral dosage forms incorporating the granules, and to methods of treating HIV using same.

### BACKGROUND OF THE INVENTION

Human immunodeficiency virus (“HIV”) is a lentivirus (a member of the retrovirus family) that causes acquired immunodeficiency syndrome (“AIDS”) (Weiss, R. A. (May 1993). “How does HIV cause AIDS?” Science 260 (5112): 1273-9. “Emerging concepts in the immunopathogenesis of AIDS”. Annu. Rev. Med. 60: 471-84) a condition in humans in which the immune system begins to fail, leading to life-threatening opportunistic infections. Infection with HIV occurs by the transfer of blood, semen, vaginal fluid, pre-ejaculate, or breast milk. Within these bodily fluids, HIV is present as both free virus particles and virus within infected immune cells. Screening of blood products for HIV has largely eliminated transmission through blood transfusions or infected blood products in the developed world. HIV infection in humans is considered pandemic by the World Health Organization (WHO). From its discovery in 1981 to 2006, AIDS killed more than 25 million people. HIV infects about 0.6% of the world's population (Joint United Nations Programme on HIV/AIDS (2006). “Overview of the global AIDS epidemic” (PDF). 2006 Report on the global AIDS epidemic. ISBN 9291734799.).

Raltegravir inhibits the catalytic activity of HIV integrase, an HIV encoded enzyme that is required for viral replication. Inhibition of integrase prevents the covalent insertion, or integration, of unintegrated linear HIV DNA into the host cell genome preventing the formation of the HIV provirus. The provirus is required to direct the production of progeny virus, so inhibiting integration prevents propagation of the viral infection. Thus, raltagrevir is known to be useful in treating HIV, and the use of raltegrevir in such treatment has received health authority approval in various countries. In the U.S., the FDA has approved the potassium salt of raltegravir as ISENTRESS®.

The chemical name for raltegravir potassium is N-[(4-Fluorophenyl)methyl]-1,6-dihydro5-hydroxy-1-methyl-2-[1-methyl-1-[[5-methyl-1,3,4-oxadiazol-2-yl)carbonyl]amino]ethyl]-6-oxo-4pyrimidinecarboxamide monopotassium salt. The empirical formula is C₂OH₂₀FKN₆O₅ and the molecular weight is 482.51. Raltegravir potassium is a white to off-white powder. It is soluble in water, slightly soluble in methanol, very slightly soluble in ethanol and acetonitrile and insoluble in isopropanol. The chemical structure of raltegravir potassium is as follows:

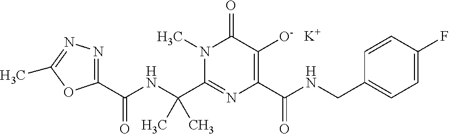 [SMILES: Cc1nnc(C(=O)NC(C)(C)c2nc(C(=O)NCc3ccc(F)cc3)c([O-])c(=O)n2C)o1 ; [K+]]

Various U.S. patents have been granted on raltegravir, related compounds and methods of using same. For example, U.S. Pat. No. 7,169,780 is directed to a genus of hydroxypyrimidinone carboxamides, which includes raltegravir. U.S. Pat. Nos. 7,217,713 and 7,435,734 are directed to methods of treating HIV and inhibiting HIV integrase, respectively, by administering compounds within the genus of U.S. Pat. No. 7,169,780. U.S. Pat. No. 7,754,731 is directed to the anhydrous crystalline potassium salt of raltegravir.

### SUMMARY OF THE INVENTION

In certain some embodiments, the present invention is directed to a pharmaceutical composition for oral administration which comprises a plurality of taste-masked granules, each of which comprises:
(i) a core granule comprising an raltegravir API and a binder, and
(ii) a taste-masking polymer composition that forms a coating on the core granule.

In some embodiments, said raltegravir API is raltegravir potassium.

In some embodiments, said raltegravir API is Form 1 of the anhydrous crystalline potassium salt of raltegravir, which is characterized by an X-ray powder diffraction pattern obtained using copper K_{α} radiation which comprises 2θ values in degrees of about 5.9, about 20.0 and about 20.6.

In some embodiments, said raltegravir API is Form 1 of the anhydrous crystalline potassium salt of raltegravir, which is characterized by an X-ray powder diffraction pattern obtained using copper K_{α} radiation which comprises 2θ values in degrees of about 5.9, about 12.5, about 20.0, about 20.6 and about 25.6.

In some embodiments, said raltegravir API is Form 1 of the anhydrous crystalline potassium salt of raltegravir, which is characterized by an X-ray powder diffraction pattern obtained using copper K_{α} radiation that is substantially similar to that displayed in FIG. 3.

In some embodiments, said raltegravir API is Form 1 of the anhydrous crystalline potassium salt of raltegravir, which is characterized by a differential scanning calorimetry curve, obtained at a heating rate of 10° C./min in a closed cup under nitrogen, exhibiting a single endotherm with a peak temperature of about 279° C.

In some embodiments, said raltegravir API is Form 1 of the anhydrous crystalline potassium salt of raltegravir, which is characterized by a differential scanning calorimetry curve, obtained at a heating rate of 10° C./min in a closed cup under nitrogen that is substantially similar to that displayed in FIG. 4.

In some embodiments, said raltegravir API is Form 2 of the anhydrous crystalline potassium salt of raltegravir, which is characterized by an X-ray powder diffraction pattern obtained using copper K_{α} radiation which comprises 2θ values in degrees of about 7.9, about 24.5 and about 31.5.

In some embodiments, said raltegravir API is Form 2 of the anhydrous crystalline potassium salt of raltegravir, which is characterized by an X-ray powder diffraction pattern obtained using copper K_{α} radiation which comprises 2θ values in degrees of about 7.9, about 13.8, about 15.7, about 24.5 and about 31.5.

In some embodiments, said raltegravir API is Form 2 of the anhydrous crystalline potassium salt of raltegravir, which is characterized by an X-ray powder diffraction pattern obtained using copper K_{α} radiation that is substantially similar to that displayed in FIG. 5.

In some embodiments, said raltegravir API is Form 2 of the anhydrous crystalline potassium salt of raltegravir, which is characterized by a differential scanning calorimetry curve, obtained at a heating rate of 10° C./min in a closed cup under nitrogen, exhibiting endotherms with peak temperatures of about 146, about 238 and about 276° C.

In some embodiments, said raltegravir API is Form 2 of the anhydrous crystalline potassium salt of raltegravir, which is characterized by a differential scanning calorimetry curve, obtained at a heating rate of 10° C./min in a closed cup under nitrogen that is substantially similar to that displayed in FIG. 6.

In some embodiments, said raltegravir API is Form 3 of the anhydrous crystalline potassium salt of raltegravir, which is characterized by an X-ray powder diffraction pattern obtained using copper K_{α} radiation which comprises 2θ. values in degrees of about 7.4, about 7.8 and about 24.7.

In some embodiments, said raltegravir API is Form 3 of the anhydrous crystalline potassium salt of raltegravir, which is characterized by an X-ray powder diffraction pattern obtained using copper K_{α} radiation which comprises 2θ. values in degrees of about 7.4, about 7.8, about 12.3, about 21.6 and about 24.7.

In some embodiments, said raltegravir API is Form 3 of the anhydrous crystalline potassium salt of raltegravir, which is characterized by an X-ray powder diffraction pattern obtained using copper K_{α} radiation that is substantially similar to that displayed in FIG. 7.

In some embodiments, said raltegravir API is Form 3 of the anhydrous crystalline potassium salt of raltegravir, which is characterized by a differential scanning calorimetry curve, obtained at a heating rate of 10° C./min in a closed cup under nitrogen, exhibiting a single endotherm with a peak temperature of about 279° C.

In some embodiments, said raltegravir API is Form 3 of the anhydrous crystalline potassium salt of raltegravir, which is characterized by a differential scanning calorimetry curve, obtained at a heating rate of 10° C./min in a closed cup under nitrogen that is substantially similar to that displayed in FIG. 8.

In some embodiments, said binder is hydroxypropyl cellulose.

In some embodiments, the weight ratio of said binder to said raltegravir API is between about 0.06 and about 0.08.

In some embodiments, said taste-masking polymer composition comprises an ethyl cellulose dispersion.

In some embodiments, the amount of raltegravir API in the composition is about 25 mg, about 50 mg, or about 100 mg, or the salt-equivalent thereof.

In some embodiments, the composition further comprises an extragranular matrix, said matrix comprising at least one flavoring agent, a disintegrant, a filler and a lubricant.

In some embodiments, said composition is a tablet or capsule.

In some embodiments, said composition is selected from Compositions A, B and C, wherein said compositions are comprised of the following:

| | Composition | | |
|---|---|---|---|
| | A | B | C |
| Raltegravir | 27 | 54 | 109 |
| Hydroxypropyl | 2 | 5 | 9 |
| cellulose | | | |
| Ethyl cellulose | 3 | 5 | 11 |
| dispersion | | | |
| Film coating blend, | 2 | 4 | 7 |
| clear Opadry | | | |
| Total Coated | 34 | 68 | 136 |
| Raltegravir Granule | | | |
| Extragranular | | | |
| Saccharin sodium | 7 | 7 | 14 |
| Sucralose | 2 | 2 | 5 |
| Banana Flavor | 5 | 5 | 9 |
| Orange Flavor | 7 | 7 | 14 |
| Monoammonium | 2 | 2 | 5 |
| Glycyrrhizinate | | | |
| Flavor Masking Neutral | 5 | 5 | 9 |
| Sodium Citrate | 1 | 1 | 2 |
| Dihydrate | | | |
| Mannitol | 154 | 120 | 240 |
| Crospovidone | 12 | 12 | 23 |
| Ferric Oxide, yellow | 1 | 1 | 2 |
| Sodium Stearyl | 2 | 2 | 5 |
| Fumarate | | | |
| Magnesium Stearate | 1 | 1 | 2 |

In some embodiments, the composition is a sachet for aqueous suspension prior to administration, further comprising at least one flavoring agent, a disintegrant, a suspending agent, a lubricant and a diluent.

In some embodiments, said composition comprises the following:

| | | Composition per sachet |
|---|---|---|
| Ingredient | Function | (mg) |
| Raltegravir | Active | 109 |
| Hydroxypropyl cellulose | Binder | 9 |
| Ethycellulose | Coating | 11 |
| Opadry I Clear | Coating | 7 |
| Crospovidone | Disintegrant | 23 |
| Mannitol | Diluent | 194 |
| Sucralose | Sweetener | 2 |
| MagnaSweet | Sweetener | 2 |
| Banana Flavor | Flavor | 9 |
| Avicel CL-611 | Suspending agent | 9 |
| Magnesium Stearate | Lubricant | 7 |

In some embodiments, said core granules have a D₅₀ of between about 130 and about 330 μm.

In some embodiments, said core granules have a D₅₀ of between about 130 and about 150 μm.

In some embodiments, said coated granules have a D₅₀ of between about 180 and about 340 μm.

In some embodiments, said coated granules have a D₅₀ of between about 170 and about 220 μm.

In some embodiments, administration of said composition to a person results in a blood plasma concentration of raltegravir displaying a Cₘₐₓ of about 3.4 μm and an AUC of about 8 μm·hr.

In other embodiments, the invention is directed to a method of treating HIV in a person in need of such treatment comprising administering to said person a pharmaceutical composition according to any of the above embodiments, wherein the dose of raltegravir API administered is about 6 mg/kg BID, or the salt equivalent thereof.

In some embodiments, said dose is determined as follows:

Patient Body Weight

| (kg) | (lbs) | Dose |
|---|---|---|
| 7 through 10 | 15 through 22 | 50 mg twice daily |
| 10 through 14 | 22 through 31 | 75 mg twice daily |
| 14 through 20 | 31 through 44 | 100 mg twice daily |
| 20 through 28 | 44 through 62 | 150 mg twice daily |
| 28 through 40 | 62 through 88 | 200 mg twice daily |
| at least 40 | at least 88 | 300 mg twice daily |

In some embodiments, said administration results in a Cₘₐₓ of about 3.4 μM and an AUC of about 8 μM-hr.

In some embodiments, said method further comprises the step of administering to said person a second anti-viral agent.

In yet other embodiments, the invention is directed to a process for preparing a pharmaceutical composition comprising the steps of:

i. granulating raltegravir API and a binder into granules by a Wurster granulating process;

ii. coating said granules with a taste-masking agent by a Wurster coating process;

iii. preparing an extragranular matrix comprising a binder, a disintegrant, a flavoring agent and a lubricant; and,

iv. compressing said granules and said matrix into a tablet.

In some embodiments, said granulating and coating steps are conducted in the same Wurster apparatus.

### BRIEF DESCRIPTION OF THE DRAWINGS

FIG. 1 displays the Wurster process unit operation.

FIG. 2 displays a block flow diagram of the manufacturing process for the raltegravir taste-masked granules.

FIG. 3 is the X-ray powder diffraction pattern for the potassium salt of raltegravir as prepared in Example 2 of U.S. Pat. No. 7,754,731.

FIG. 4 is the DSC curve for the potassium salt of raltegravir as prepared in Example 2 of U.S. Pat. No. 7,754,731.

FIG. 5 is the X-ray powder diffraction pattern for the potassium salt of raltegravir as prepared in Example 4 of U.S. Pat. No. 7,754,731.

FIG. 6 is the DSC curve for the potassium salt of raltegravir as prepared in Example 4 of U.S. Pat. No. 7,754,731.

FIG. 7 is the powder X-ray diffraction pattern for the potassium salt of raltegravir as prepared in Example 5 of U.S. Pat. No. 7,754,731.

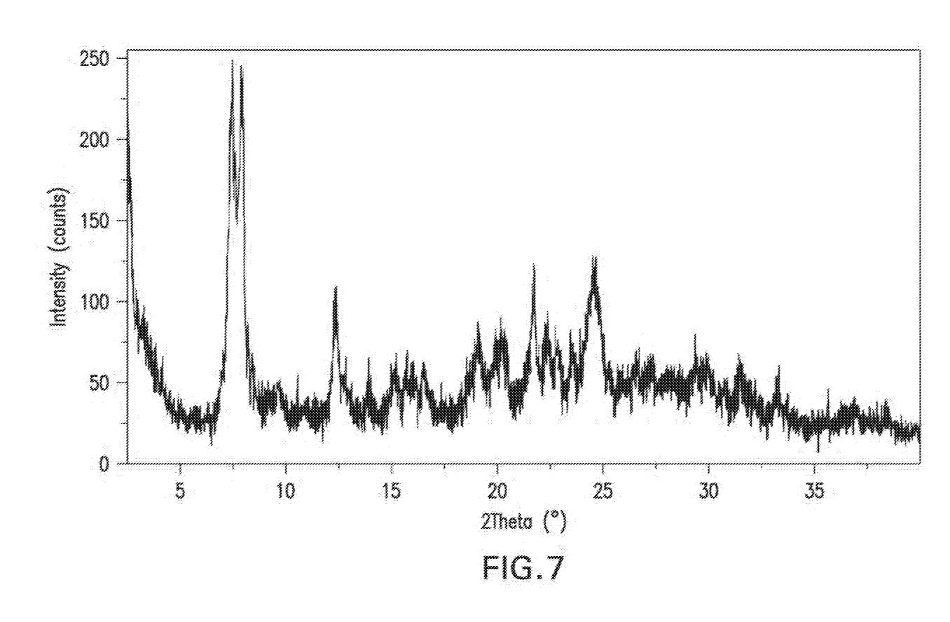

FIG. 8 is the DSC curve for the potassium salt of raltegravir as prepared in Example 5 of U.S. Pat. No. 7,754,731.

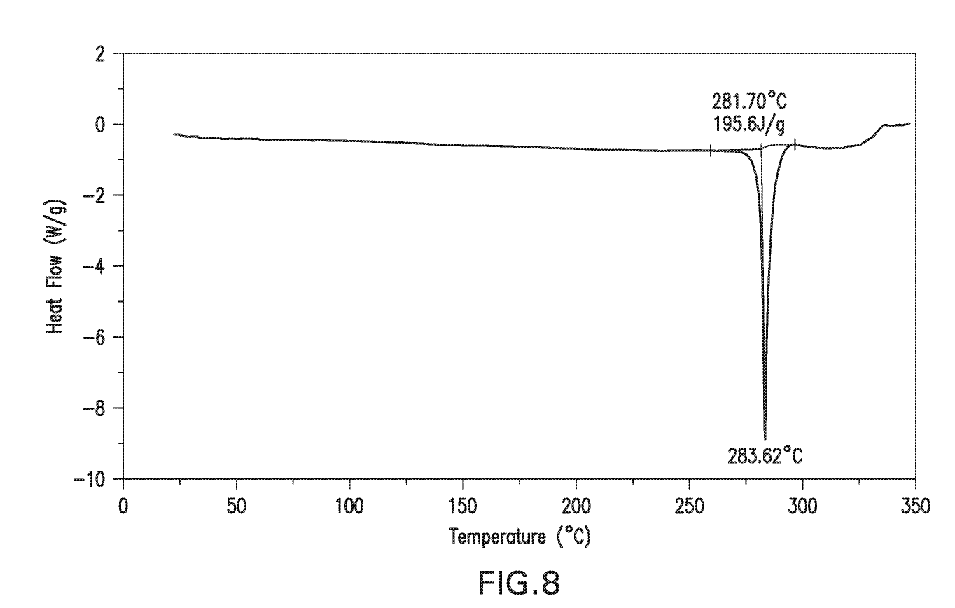

FIG. 9 displays a block flow diagram of the manufacturing process for the raltegravir chewable tablets.

FIG. 10 displays a block flow diagram of the manufacturing process for the raltegravir sachet product.

FIG. 11 displays the effect of product temperature on the particle size distribution of uncoated granules for several process conditions.

### DETAILED DESCRIPTION OF THE INVENTION

In pediatric populations, chewable tablets or sachets may be advantageous formulations for ease of administration. Raltegravir can be provided as a crystalline, anhydrous, potassium salt. The API is a fine particle of mean size ˜30 microns and has solubility in water of 71 mg/ml. The pH of this solution is 9.3. As a consequence of this high pH, raltegravir potassium is reported to result in a particularly unpleasant taste sensation when contact is made in the oral cavity. It is also noted that the adult tablet formulation required film coating for taste-masking purposes. Thus, the challenge facing formulators of such a product targeting the pediatric HIV population was to obtain a chewable tablet or sachet containing a safe and therapeutically effective amount of raltegravir that avoids the generation of the noxious taste sensation inherent to the active.

### Initial Prototypes

Early pediatric development studies utilized the pH sensitive dissolution of Eudragit EPO, a methacrylate polymer system, to provide a matrix for drug release. The matrix was prepared by dry granulation utilizing roller compaction. It was found that a 2:1 ratio of drug to polymer was required for the dosage form to have the desired release attributes.

The initial prototype pediatric formulation is displayed in Table 1. As shown in the table, the formulators settled on an Eudragit level of 50 mg as being sufficient to assure adequate taste-masking in this 100 mg raltegravir tablet.

TABLE 1

| Ingredient | Function | Mg/tablet | |
|---|---|---|---|
| Raltegravir (003E) | Active | 108.6 - potassium | |
| | | salt equivalent to | |
| | | 100 mg free base. | |
| Aspartame | Sweetener | 5.6 | |
| Banana flavor | Flavor | 2.8 | |
| Microcrystalline cellulose | Filler | 26 | |
| Monoammonium | Sweetener | 2.8 | |
| glycyrrhizinate | | | |
| Basic butylated methacrylate | Taste-masking agent | 50 | |
| co-polymer Eudragit EPO ™ | | | |
| Magnesium stearate | Lubricant | 4.2 | |
| Extragranular | | | |
| Aspartame | Sweetener | 5 | |
| Banana flavor | Flavor | 5 | |
| Crospovidone | Disintegrant | 25 | |
| Mannitol | Filler | 250 | |
| Magnesium Stearate | Lubricant | 15 | |
| Tablet weight | | 500 | mg |

In pediatric populations, Eudragit is associated safety risk of exposure to residual monomer. Upon review, the 50 mg level of Eudragit in this formulation was deemed too high for application in a pediatric formulation. Thus, an alternate manufacturing process was sought to bring the level of polymer required for taste-masking to a level acceptable for pediatric dosing.

The invention involves the development of a taste-masked granule of raltegravir utilizing Wurster granulation and coating technology. The Wurster granulation process utlizes pure bulk API to provide a granule with high drug loading. Processing is designed to provide minimal agglomeration to facilitate mouth feel for this pediatric application.

Pharmaceutical compositions targeted for administration to a pediatric population often take the form of chewable tablets or suspended particulate, in view of the difficulty faced by a young person in swallowing a whole tablet. Both of these compositions start with some form of granules containing the active ingredient. In this case, to obtain sufficient taste-masking, the granules would have to be coated. The high API loading required to achieve therapeutic effect and the need to avoid fine particles that might avoid coating led to the conclusion that the granulation and coating processes should result in a relatively well controlled granule size distribution.

Wurster fluid bed granulation is a manufacturing process that allows a precision coating to be applied to particles, as described in WO2006/052503, which is incorporated in its entirety.

### Wurster Process

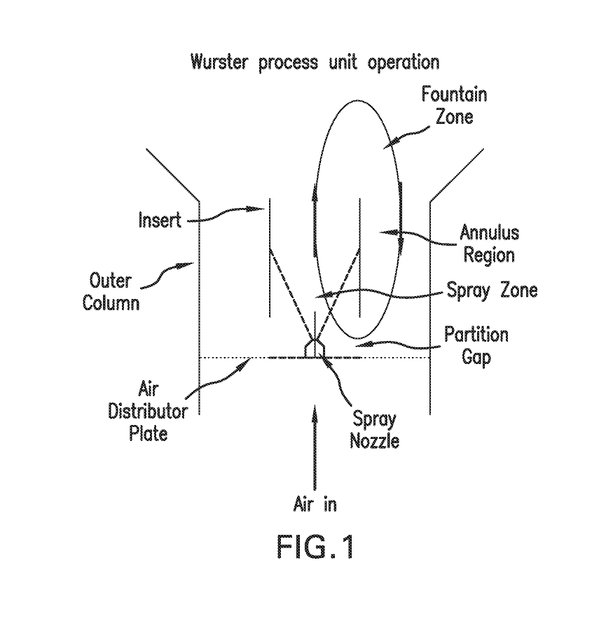

The Wurster unit operation is commonly used for applying a layer of coating over a substrate in the pharmaceutical industry. The Wurster process unit operation is displayed in FIG. 1. The Wurster unit consists of two concentric cylinders, the insert and the annulus, above a distributor plate. The solids to be coated are loaded in the annulus. Upon initiation of airflow, the solids from the annulus pass through the partition gap and are pneumatically conveyed into the insert. The coating solution is sprayed through the nozzle at the distributor plate and coats the solids flowing in the insert. The solids lose their momentum in the fountain zone and fall back into the annulus where they move downward and back into the insert. The deposited coat dries mainly in the insert and fountain zone. The recirculation is continued until the desired coat weight is achieved.

In some aspects, the present invention is directed to a Wurster granulation process, which is a process for granulating pharmaceutical ingredients using a Wurster unit operated above the mass transfer limit. The invention also encompasses a two-step process that encompasses first, granulation and then coating for the preparation of taste-masked or controlled release API formulations using the Wurster granulation process. The invention also allows for the granulation of materials of different physical characteristics using the Wurster granulation process, e.g., beads/agglomerates/granules (mean diameter less than 300 μm) with powders (mean diameter less than 150 μm).

The Wurster granulation process has distinct advantages over the conventional high-shear and fluid-bed granulation processes. These advantages over the conventional granulation process are:

(1) The recirculation in the Wurster granulation process provides uniform distribution of the granulating solution to the solid particles, resulting in uniform and homogeneous granulation. The distribution of granulating solution onto the solid particles in the high-shear and fluid-bed granulation processes is restricted to the event when the solid particles are exposed to the spray zone. This is due to the narrow spray zone as compared to the entire solids bed in the conventional processes. In addition, the exposure of solid particles being exposed to the narrow spray zone is uncontrolled, and thus chaotic in the conventional processes as compared to the ordered recirculation process in the Wurster granulation process. Thus, the Wurster granulation process due to its orderly recirculation imparts uniform granulation characteristics and better control of granulation as compared to that in the conventional granulation processes.

(2) The uniform granulation enables tighter control of the granule size distribution for special applications such as controlled release or taste-masked technology.

(3) The Wurster unit operation allows a single vessel process for taste-masking and controlled release applications where the granulation step can be followed by incorporation of taste-masking or controlled release coat by conducting coating in the same Wurster unit.

(4) Wurster granulation provides the ability to quantify and scale up the granulation process using chemical engineering principles. The granulation kinetics can be easily related to heat/mass transfer and hydrodynamic characteristics since application of these principles has already been demonstrated for coating processes in the Wurster unit. Geometric scale-up issues can be minimized by utilizing multiple development-scale inserts in the commercial-scale Wurster unit.

(5) Wurster granulation has the potential for providing granules with better attrition resistance than the high shear granulation/fluid-bed drying processes since the granules are prepared under high velocity/impact conditions in the Wurster unit operation.

(6) The Wurster process facilitates on-line control of granule size using existing technology since sticking issues observed in high-shear and fluid-bed granulations are not present in the annulus region of the Wurster unit.

Due to the fine particle size of the starting active and the desire to provide a concentrated intermediate for application to formulation platforms, a granulation step was first required to densify the API and provide granules for subsequent coating. Early development studies looked at the preparation of granules with API content of 75% and a filler of either lactose or mannitol with a binder. Hydroxypropyl cellulose SL was selected as the binder as it is a low viscosity grade of hydroxypropyl cellulose (HPC) which would impart rapid release of drug from the granules. The objective of the granulation step is to provide a substrate for polymer coating. Wurster granulation was selected as the manufacturing process as it provides a relatively tight particle size distribution with controlled granule growth. Early studies found that pure API could be coated with 6 to 8% HPC with controlled granule growth as polymer loading increased. Eight percent HPC-SL was selected as the polymer level for the granulation. This level provided strong granules for polymer coating for taste-masking. The target granule size for the granulation step was selected to provide ease of precision coating with the taste-masking polymer, to provide controlled granule growth to minimize the presence of large particles in excess of 400 microns which would lead to sample grittiness, and to provide a particle size range which would blend uniformly with the extragranular excipients for the preparation of the chewable tablet as well as the oral granules for suspension.

### Definitions

As used herein, “API” means active pharmaceutical ingredient.

As used herein, the term “raltegravir API” means raltegravir free base or a pharmaceutically acceptable salt thereof.

As used herein, the term “taste-masking agent” means, for example, polymethacrylate (EUDRAGIT), hydropropylmethylcellulose (HMPC), Hydroxypropylcellulose, (HPC) and vinyl pyrrolidone-vinyl acetate co-polymer (PLASDONE).

As used herein, the term “sweetening agent” means, for example, sugar and aspartame.

As used herein, the term “flavoring agent” means for example, artificial flavor, such as artificial cherry flavor.

As used herein, the term “bulking agent” means, for example, mannitol, lactose, starch and calcium phosphate.

As used herein, the term “binder” means, for example, hydroxypropyl cellulose (HPC) or hydroxypropyl methyl cellulose (HPMC).

As used herein, the term “granulation solution” means, for example, aqueous solution of “binder” agents as defined above.

As used herein, the term “disintegrant” refers to a substance added to the dosage form to help it break apart (disintegrate) and release the medicinal agent(s). Suitable disintegrants include: microcrystalline celluloses and cross-linked celluloses such as sodium croscarmellose; starches; “cold water soluble” modified starches such as sodium carboxymethyl starch; natural and synthetic gums such as locust bean, karaya, guar, tragacanth, and agar; cellulose derivatives such as methylcellulose and sodium carboxymethylcellulose; alginates such as alginic acid and sodium alginate; clays such as bentonites; and effervescent mixtures. Preferred disintegrants comprise croscarmellose sodium, starch, sodium starch glycolate, crospovidone and croscarmelose sodium and microcrystalline cellulose. The amount of disintegrant in the composition can range from about 2% to about 12% by weight of the composition, more preferably from about 3.5% to about 6% by weight.

As used herein for solid oral dosage forms of the present invention, the term “lubricant” refers to a substance added to the dosage form to enable the tablet after it has been compressed, to release from the mold or die by reducing friction or wear. Suitable lubricants include metallic stearates such as magnesium stearate (vegetable grade), calcium stearate or potassium stearate; stearic acid; high melting point waxes; and water soluble lubricants such as sodium chloride, sodium benzoate, sodium acetate, sodium oleate, polyethylene glycols, and d′l-leucine. Preferred lubricants comprise magnesium stearate, stearic acid and talc. The amount of lubricant in the composition can range from about 0.1% to about 2% by weight of the composition, preferably about 0.5% by weight.

As used herein, the term “Wurster process” or “Wurster granulation process,” refers to a process for granulating particles by subjecting the particles to a repeated circulating movement comprising:

a non-rotating upward pneumatical movement from a starting area inside a vertical granulation pipe, wherein said particles are subjected to a spray of droplets of granulation solution, and

a downward movement outside said pipe and a horizontal movement towards the starting area for said pneumatical movement wherein said process is operated above the mass transfer limit to facilitate the agglomeration of the particles.

As used herein, the term “Wurster coating process” means a process for granulating particles by subjecting the particles to a repeated circulating movement comprising:

a non-rotating upward pneumatical movement from a starting area inside a vertical granulation pipe, wherein said particles are subjected to a spray of droplets of coating solution, and

a downward movement outside said pipe and a horizontal movement towards the starting area for said pneumatical movement.

As used herein, the terms “D₁₀,” “D₅₀,” and “D₉₀” mean the arithmetic mean the particle size that marks the subscripted percent of particles in the distribution. Thus, for example, the value given for D₅₀ is the particle size below which 50% of all particles reside in the distribution. These values are expressed in μm.

As used herein, the term “administration” and variants thereof (e.g., “administering” a compound) means providing the compound to the individual in need of treatment or prophylaxis. When a compound is provided in combination with one or more other active agents (e.g., antiviral agents useful for treating or prophylaxis of HIV infection or AIDS), “administration” and its variants are each understood to include provision of the compound and other agents at the same time or at different times. When the agents of a combination are administered at the same time, they can be administered together in a single composition or they can be administered separately.

As used herein, the term “composition” is intended to encompass a product comprising the specified ingredients, as well as any product which results, directly or indirectly, from combining the specified ingredients.

As used herein, “pharmaceutically acceptable” is meant that the ingredients of the pharmaceutical composition must be compatible with each other and not deleterious to the recipient thereof.

As used herein, the term “subject” refers to an animal, preferably a mammal, most preferably a human, who has been the object of treatment, observation or experiment.

As used herein, the term “effective amount” means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HIV integrase (wild type and/or mutant strains thereof) and thereby elicit the response being sought (i.e., an “inhibition effective amount”). When the active compound (i.e., active ingredient) is administered as the salt, references to the amount of active ingredient are to the free form (i.e., the non-salt form) of the compound.

As used herein, the term “bioequivalent” means an alternate formulation, dosage strength, or dosing regimen that, after administration to a patient results in a pharmacokinetic attribute (e.g., Cₘₐₓ, AUC) that is within the range from about 80% of the reference parameter to about 125% of the reference parameter. Thus, for example, where a reference formulation results in an average Cₘₐₓ of 10 μm, after administration a formulation that results in a Cₘₐₓ of between about 8 μm and about 12.5 μm would be bioequivalent to the reference formulation.

As used herein, the term “Cₘₐₓ” means the mean maximum plasma concentration of a drug achieved in a patient after that drug has been administered to the patient.

As used herein, the term “AUC” means the area under the plasma drug concentration versus time curve. It is a measure of drug exposure.

As used herein, the term “salt equivalent” refers to that weight quantity of a salt of raltegravir that is comprised of the stated weight quantity of the free base form of raltegravir.

As used herein, the term “400 mg tablet” means the commercially available Isentress 400 mg tablet heretofore approved for adult dosing.

As used herein, the term “chewable tablets” means the tablets whose compositions are provided in Table 5.

### Tablet Process

The preparation of the chewable raltegravir tablets is broadly divided into two steps:

1) generation of the coated raltegravir granules; and,

2) formation of chewable tablets that incorporate the coated raltegravir granules.

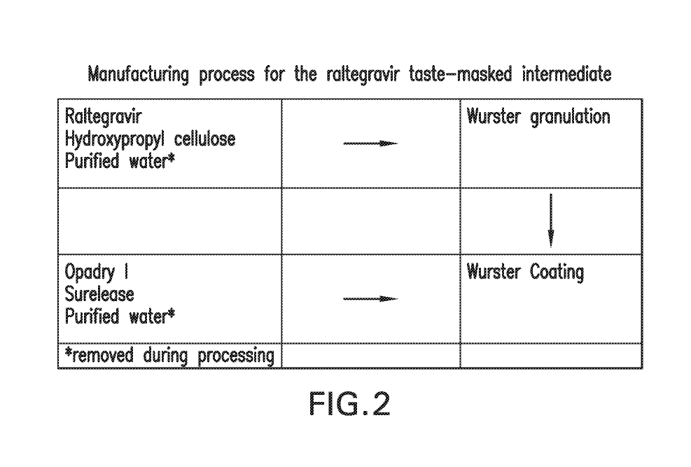

The generation of the coated raltegravir granules by the Wurster process is comprised of a granulation step by a Wurster granulation process, and a coating step by a Wurster coating process, as illustrated by the block flow diagram displayed in FIG. 2.

### API Granulation

Table 2 displays the raw materials used in preparation of the granules.

TABLE 2

| | Quantity |
|---|---|
| Ingredient | mg/g |
| Raltegravir potassium | 920.0** |
| Hydroxypropyl Cellulose, Compendial [HPC-SL] | 80.0 |
| Purified Water, USP (removed during processing) | N/A |
| | 1000 |

**Equivalent to 847.1 mg of raltegravir free base (1.086 conversion factor)

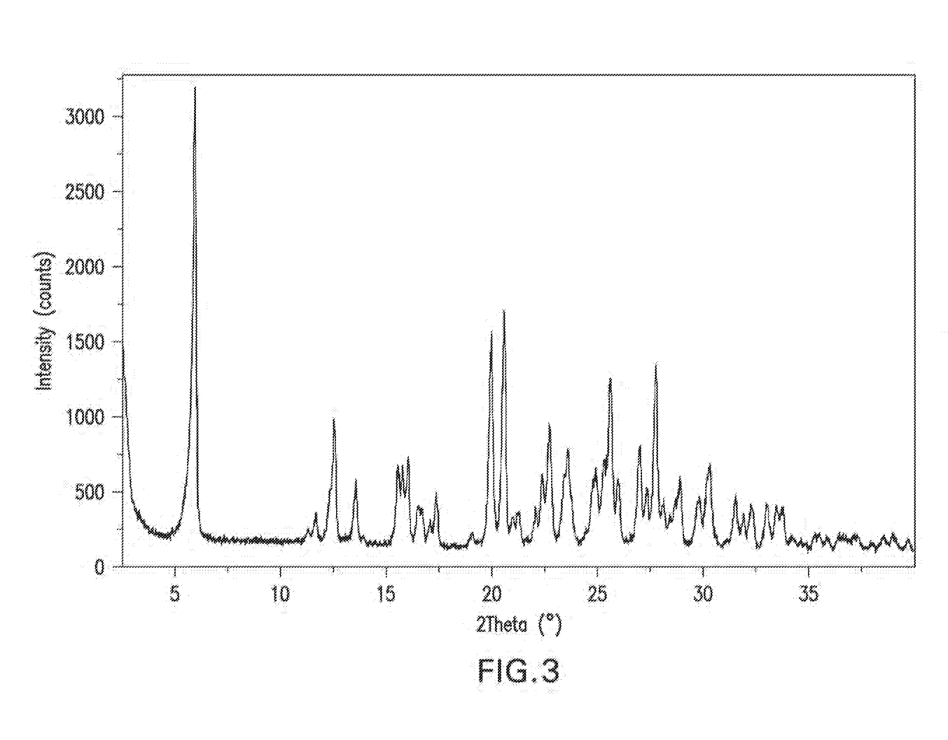

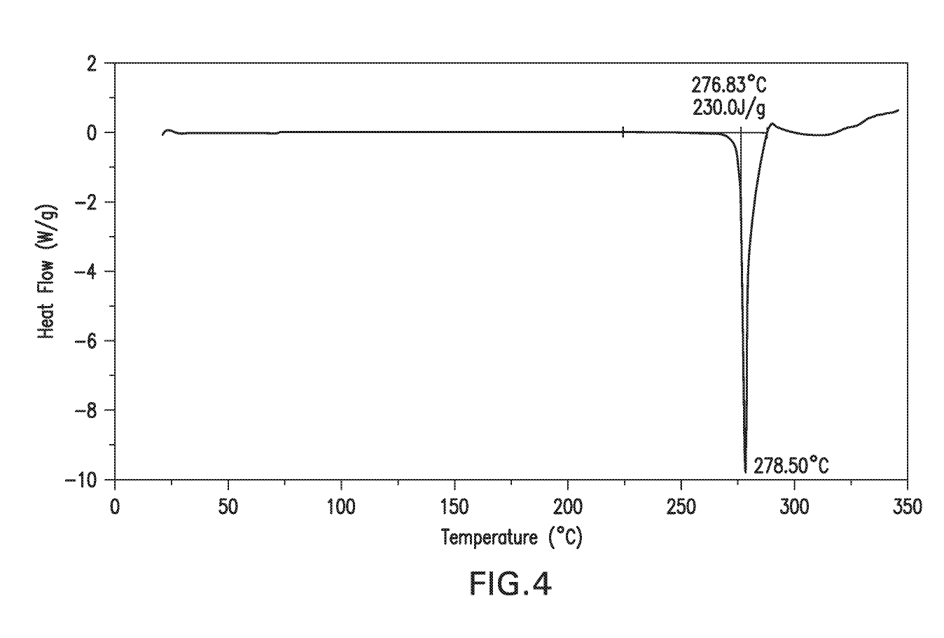

The API raw material is anhydrous raltegravir potassium salt in the crystalline form that is labelled as Form 1 in U.S. Pat. No. 7,754,731. A procedure for the preparation of this form of the potassium salt is provided in Example 2 (col. 46, line 7-col. 47, line 22), which is incorporated by reference. FIGS. 3 and 4 in this specification (FIGS. 1 and 2 in U.S. Pat. No. 7,754,731) display the characteristic powder x-ray diffraction (“PXRD”) pattern (using copper K α radiation) and differential scanning calorimetery (“DSC”) curve, respectively, that are characteristic to Form 1 of the raltegravir potassium salt. The PXRD pattern displays characteristic peaks at about 5.0, 20.0 and 20.6 degrees 2θ. The DSC curve displays a single sharp exotherm at about 279 deg. C (heat of fusion=about 230 J/gm).

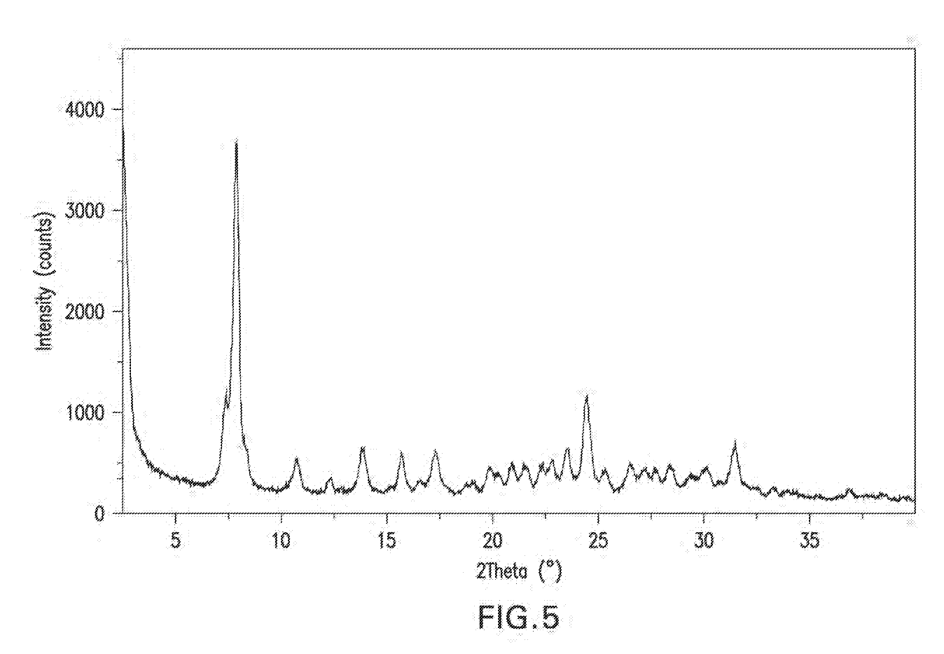

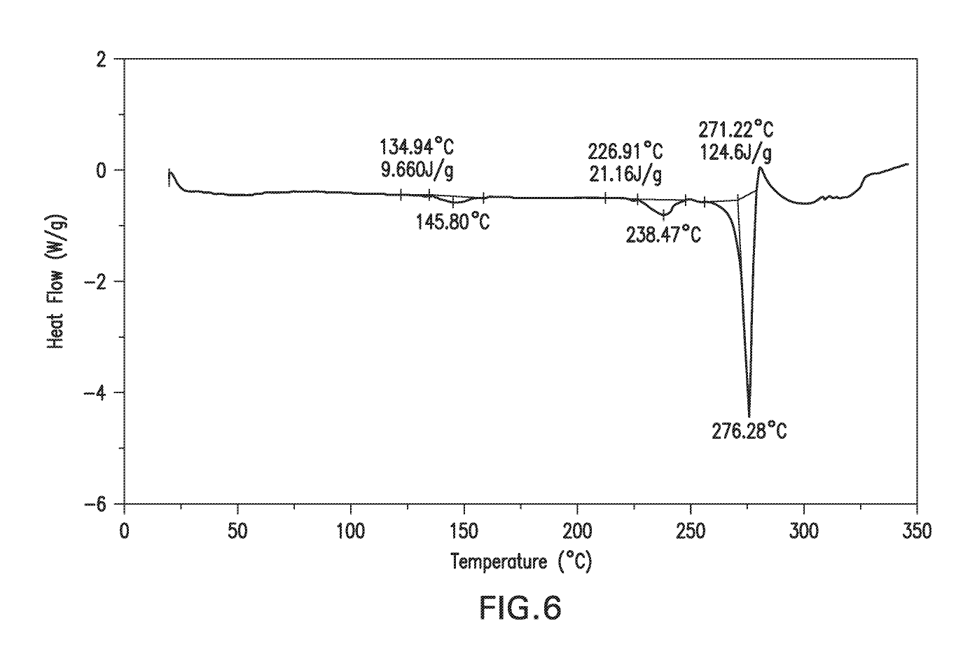

The invention further encompasses compositions comprising alternate crystalline forms of raltegravir potassium. Two other such forms are disclosed in U.S. Pat. No. 7,754,731, in which they are labeled Form 2 and Form 3. The synthesis of Forms 2 and 3 are disclosed in Examples 4 and 5, respectively (see col. 47, line 61-col. 49, line 37), which are incorporated by reference. Physicochemical characterizations by way of PXRD spectra and DSC profiles of Forms 2 and 3 are displayed here in FIGS. 3-6 (in U.S. Pat. No. 7,754,731, FIGS. 1-4). The invention also encompasses compositions in which the raltegravir is in other salt forms on the free base form.
Equipment used in preparation of the granules was as follows:
Wurster Apparatus: Glatt® Model GPCG-3

Bowl=7″ Wurster Bowl with Wurster Insert

Baseplate=D-7

Secondary Screen=325 mesh

Wurster Insert Height=˜30 mm

Filter Bag=2-5 micron (2 sock)

Peristaltic Pump Head=7516-10

Tubing Size=16

Liquid Tip/Air Cap=1.2 mm/2.6 mm
The process parameters in the granulation step were set as follows:

Atomization Air Pressure=20 psi

Target Inlet Air Temperature=˜40 C

Target Exhaust RH=˜70-80%

Target Solution Delivery Rate=˜15-18 mL/min
The granulation step proceeded according to the following:

Prepare a 10% (w/w) HPC-SL solution by adding HPC-SL in hot USP purified water. The solution was mixed using Lightnin® mixer until completed dissolved. The column was pre-heated to ˜40 C. API was charged into bowl (around insert), avoiding powder contact with spray nozzle to prevent clogging during granulation. Airflow was introduced into column to fluidize powder. HPC-SL solution spray into column at a rate of 10 mL/min The pump was gradually released to 15-18 mL/min. Once target solution delivery amount had been achieved, spraying stopped. Dry granulation continued until final exhaust % RH equaled the initial exhaust % RH.

### Granule Coating

The second step in the generation of the coated granules is the Wurster coating process of the uncoated raltegravir granules. The raw materials used in this coating step are shown in Table 3.

TABLE 3

| | Quantity |
|---|---|
| Ingredient | mg/g |
| Raltegravir 847 mg/g, Uncoated | 869.6 |
| Ethylcellulose, Aqueous Dispersion, Clear [Surelease ® | 90.00 |
| E-7-19010] | |
| Film Coat Blend, Powder, Clear [Opadry ® YS-1-19025-A] | 60.00 |
| Purified Water, USP (removed during processing) | N/A |

The equipment used in the coating step is as follows:
Wurster Apparatus: Glatt® Model GPCG-3

Bowl=7″ Wurster Bowl with Wurster Insert

Baseplate=D-7

Secondary Screen=325 mesh

Wurster Insert Height=˜30 mm

Filter Bag=2-5 micron (2 sock)

Peristaltic Pump Head=7516-10

Tubing Size=16

Liquid Tip/Air Cap=1.2 mm/2.6 mm
The process parameters were set as shown below:

Atomization Air Pressure=25 psi

Target Inlet Air Temperature=˜60 C

Target Exhaust RH=˜40-50%

Target Solution Delivery Rate=˜12-15 mL/min
The coating step proceeded according to the following:

A 10% (w/w) Surelease®/Opadry® I dispersion was prepared by first adding Opadry® I clear into USP purified water. The dispersion was mixed using Lightnin® mixer until completed dissolved. Surelease® aqueous dispersion was added into the Opadry® I clear mixture and stirred until uniform (approximately 15-20 minutes). The column was pre-heated to ˜60 C. The granulation was charged into bowl (around insert), avoiding powder contact with spray nozzle to prevent clogging during coating. Airflow was introduced into column to fluidize granulation. Surelease® Opadry® I dispersion was sprayed into column at a spray rate of 8 mL/min. The pump setting was gradually increased to 12-15 mL/min Once target solution delivery amount had been achieved, spraying was stopped. Dry coating continued until final exhaust % RH equaled the initial exhaust % RH.

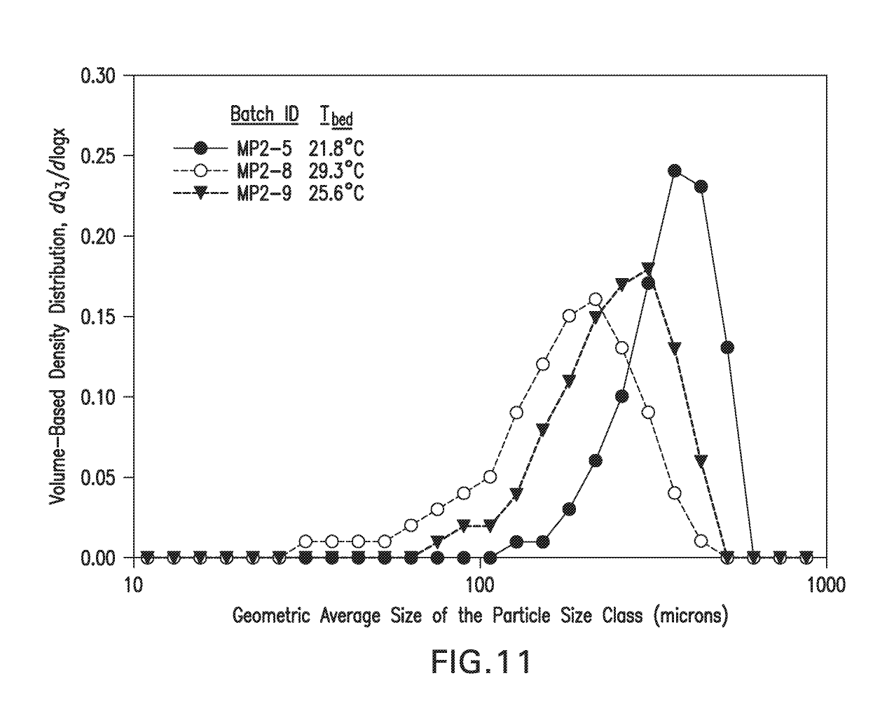

FIG. 11 dislays batch particle size distributions of raltegravir granules that resulted from the above-described process.

### Chewable Tablet Compositions

Using raltegravir granules produced by the above processes, a variety of prototype tablet compositions were generated, as displayed in Table 4.

TABLE 4

| NB 71788- | NB 71788- | NB 71788- | NB 71788- | NB 71788- |
|---|---|---|---|---|
| 135 | 147 | 165 | 189 | 205 |

| | |
|---|---|
| Materials | Percentage % |

INTRAGRANULAR

| | | | | | |
|---|---|---|---|---|---|
| Raltegravir K Salt | 27.15 | 21.72 | 6.79 | 6.79 | 21.72 |
| Aspartame | 0.5 | 0.5 | 1 | 1 | 1.12 |
| Grape Flavor | 0.5 | 0.5 | 0.5 | — | 0.56 |
| Banana Flavor | — | — | — | 0.5 | — |
| MagnaSweet 135 | — | — | 0.5 | 0.5 | 0.56 |
| Eudragit EPO | — | — | 3.13 | 3.13 | 10 |
| Avicel PH102 | 7.06 | 8.265 | 23.04 | 23.04 | 5.2 |
| Pearlitol 200SD | — | 8.265 | — | — | — |
| Magnesium Stearate | 0.5 | 0.75 | 0.75 | 0.75 | 0.84 |

EXTRAGRANULAR

| | | | | | |
|---|---|---|---|---|---|
| Aspartame | 0.75 | 0.5 | 1 | 1 | 1 |
| Grape Flavor | 1.25 | 1 | 1 | — | 1 |
| Banana Flavor | — | — | — | 1 | — |
| Crospovidone | 7.5 | 5 | 5 | 5 | 5 |
| Spray-Dried Lactose | — | — | — | — | 25.75 |
| (Fast Flo) | | | | | |
| Pearlitol 200SD | 54.04 | 52.5 | — | 55.79 | 22.75 |
| Pearlitol 500DC | — | — | 56.29 | — | — |
| Magnesium Stearate | 0.75 | 1 | 1 | 0.5 | 0.5 |
| Sodium Stearyl | — | — | — | 1 | 1 |
| Fumarate | | | | | |
| Comments | Use of 7.5% | Preparation of | Use of | Use of banana | Replaced a |
| | crospovidone | ODT without | Pearlitol | flavor instead | portion of the |
| | rather than | Eudragit EPO | 500DC instead | of grape flavor | extragranular |
| | the usual 5% | in formulation | of Pearlitol | | mannitol with |
| | crospovidone | | 200SD in an | | spray-dried |
| | | | effort to match | | lactose (Fast |
| | | | the particle | | Flo) to |
| | | | size of the | | evaluate tablet |
| | | | primary | | hardness and |
| | | | extragranular | | disintegration |
| | | | excipient with | | time |
| | | | the particle | | |
| | | | size of the | | |
| | | | granules (~500 μm) | | |
| Results | Slow | Low tablet | Slow | Acceptable | Slow |
| | disintegration | hardness | disintegration | tablet hardness | disintegration |
| | time | (1.6-1.9 kP) | time | (2.0 kP). | time |
| | (23-27 sec) | Slow | (24-26 sec) | Acceptable | (22-25 sec) |
| | | disintegration | | disintegration | “Gritty” mouth |
| | | time | | time (15 sec). | feel |
| | | (105-112-sec) | | Acceptable | |
| | | | | taste and | |
| | | | | mouth feel | |

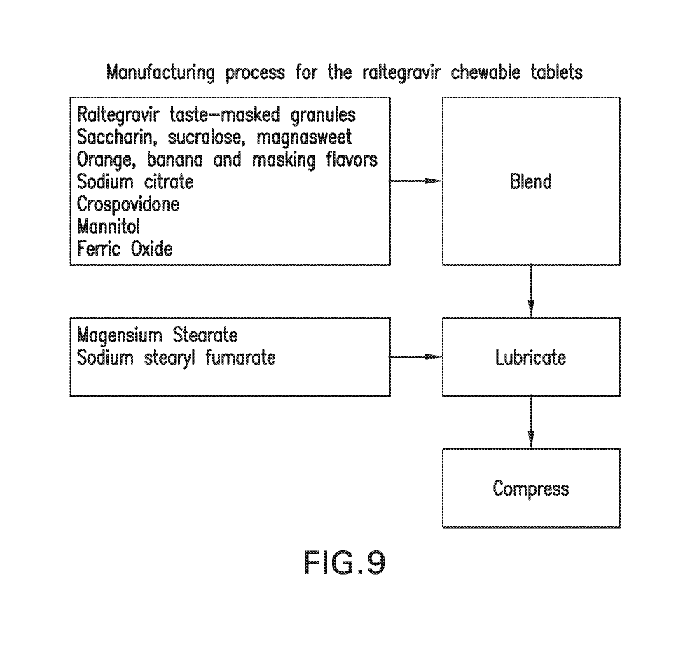

As indicated in the row labeled “Results” in the above table, NB 71788-189 had the best characteristics, and was used as the basis for further development. FIG. 9 is a block flow diagram of the process for preparing tablets that incorporate the raltegravir granules. The extragranular excipients are added as screened powders in a blending step with coated granules prior to compression. Using the processes and equipment analogous to that described above, the final three chewable tablet compositions were prepared, corresponding to 25 mg, 50 mg and 100 mg potency tablets whose composition is displayed in Table 5.

TABLE 5
Composition

| | | 25 mg | 50 mg | 100 mg |
|---|---|---|---|---|
| | | potency | potency | potency |
| Ingredient | Function | mg/unit | mg/unit | mg/unit |
| Raltegravir | Active | 27.16 | 54.3 | 108.6 |
| Hydroxypropyl | Binder | 2.362 | 4.718 | 9.438 |
| cellulose | | | | |
| Ethyl cellulose | Taste-masking | 2.657 | 5.316 | 10.633 |
| dispersion | | | | |
| Film coating blend, | Pore former, taste- | 1.771 | 3.544 | 7.088 |
| clear Opadry | masking | | | |
| Total Coated | Taste-masked | 33.95 | 67.89 | 135.8 |
| Raltegravir Granule | active | | | |
| Extragranular | | | | |
| Saccharin sodium | Sweetener | 6.999 | 6.999 | 14.0 |
| Sucralose | Sweetener | 2.334 | 2.334 | 4.667 |
| Banana Flavor | Flavor | 4.667 | 4.667 | 9.334 |
| Orange Flavor | Flavor | 6.999 | 6.999 | 14.0 |
| Monoammonium | Sweetener | 2.334 | 2.334 | 4.667 |
| Glycyrrhizinate | | | | |
| Flavor Masking | Flavor | 4.667 | 4.667 | 9.334 |
| Neutral | | | | |
| Sodium Citrate | Flavor | 1.167 | 1.167 | 2.333 |
| Dihydrate | | | | |
| Mannitol | Filler | 153.84 | 119.76 | 239.6 |
| Crospovidone | Disintegrant | 11.67 | 11.67 | 23.34 |
| Ferric Oxide, yellow | Colorant | 1.167 | 1.167 | 2.333 |
| Ferric Oxide, red | Colorant | 0.14 | 0.14 | 0.28 |
| Sodium Stearyl | Lubricant | 2.334 | 2.334 | 4.667 |
| Fumarate | | | | |
| Magnesium Stearate | Lubricant | 1.167 | 1.167 | 2.334 |
| Total Tablet weight | | 233.3 | 233.3 | 466.6 |

All three of the tablet formulations in Table 5 had acceptable quality attributes and demonstrated an acceptable processing range. Peak product moisture (“LOD”) ranged from 0.5-1.2%, the D₅₀ after granulation ranged from approximately 130-150 μm and 170-220 μm after coating. Small ranges in these process responses indicate excellent reproducibility of the process.

A clinical trial is under way in which the compositions in Table 5 will be administered to a pediatric population. Appropriate combinations of the 25, 50 and 100 mg raltegravir compositions will be administered to achieve a dosing of 6 mg/kg. This dose will be administered twice per day (BID).

Table 6 displays the physical characteristics of fluid bed uncoated and coated granules produced in a scaled-up process.

TABLE 6

| | | Inlet | Liquid-to-Air | Spray Rate- | Average | | | | | |
|---|---|---|---|---|---|---|---|---|---|---|
| | | Air | Ratio Squared, | to-Pressure | Product | Density | | Granule Size Distribution | | |
| Batch | Process | Temp. | S/Q (g/min/ | Ratio, S/P² | Bed Temp. | (g/mL) | | (μm) | | |
| ID | Step | (° C.) | cu. ft. air) | (g/min/bar²) | (° C.) | Bulk | Tap | D₁₀ | D₅₀ | D₉₀ |
| MP2-1 | Granulation | 56 | 0.3 | 4.5 | 26.7 | 0.44 | 0.53 | 156.5 | 272.3 | 410.9 |
| | Coating | 80 | 0.4 | 4.2 | 30.5 | 0.58 | 0.67 | 168.8 | 280 | 434 |
| MP2-2 | Granulation | 70 | 0.2 | 2.4 | 37 | 0.48 | 0.58 | 121.7 | 240 | 391.9 |
| | Coating | 70 | 0.3 | 6.7 | 32.7 | 0.51 | 0.61 | 140.1 | 228.5 | 342.1 |
| MP2-3 | Granulation | 53 | 0.4 | 2.4 | 21.6 | 0.51 | 0.64 | 43.3 | 134.2 | 256.1 |
| | Coating | 70 | 0.5 | 1.6 | 23.9 | 0.45 | 0.53 | 159.8 | 318.8 | 511.9 |
| MP2-4 | Granulation | 70 | 0.2 | 6.7 | 36.4 | 0.43 | 0.52 | 152.4 | 251.9 | 382.6 |
| | Coating | 70 | 0.5 | 1.6 | 26 | 0.51 | 0.61 | 191.6 | 311.1 | 439.1 |
| MP2-5 | Granulation | 53 | 0.4 | 6.7 | 21.8 | 0.48 | 0.56 | 196.4 | 328.9 | 452.1 |
| | Coating | 70 | 0.3 | 6.7 | 32.5 | 0.55 | 0.64 | 137.3 | 254.3 | 396.3 |
| MP2-6 | Granulation | 53 | 0.2 | 6.7 | 28.5 | 0.5 | 0.6 | 135.9 | 264.8 | 401.5 |
| | Coating | 90 | 0.3 | 1.6 | 37.8 | 0.54 | 0.63 | 142.3 | 230 | 362.6 |
| MP2-7 | Granulation | 70 | 0.4 | 2.4 | 28.1 | 0.53 | 0.64 | 69.3 | 174.4 | 287.5 |
| | Coating | 90 | 0.3 | 1.6 | 38.8 | 0.56 | 0.67 | 108.3 | 180 | 288.4 |
| MP2-8 | Granulation | 70 | 0.4 | 6.7 | 29.3 | 0.48 | 0.58 | 71.7 | 166.3 | 278.1 |
| | Coating | 90 | 0.5 | 6.7 | 31.3 | 0.48 | 0.57 | 125.7 | 219.7 | 346 |
| MP2-9 | Granulation | 56 | 0.3 | 4.5 | 25.6 | 0.44 | 0.55 | 112.6 | 223.8 | 347 |
| | Coating | 80 | 0.4 | 4.2 | 33.4 | 0.48 | 0.59 | 110.6 | 194.6 | 309.9 |
| MP2-10 | Granulation | 53 | 0.2 | 2.4 | 26.6 | 0.468 | 0.559 | 141.7 | 279.8 | 413.6 |
| | Coating | 90 | 0.5 | 6.7 | 32.4 | 0.461 | 0.552 | 188.7 | 335 | 455.8 |

The range of D₅₀ for uncoated granules was from about 130 μm to about 330 μm. The range of D₅₀ for coated granules was from about 180 μm to about 340 μm.

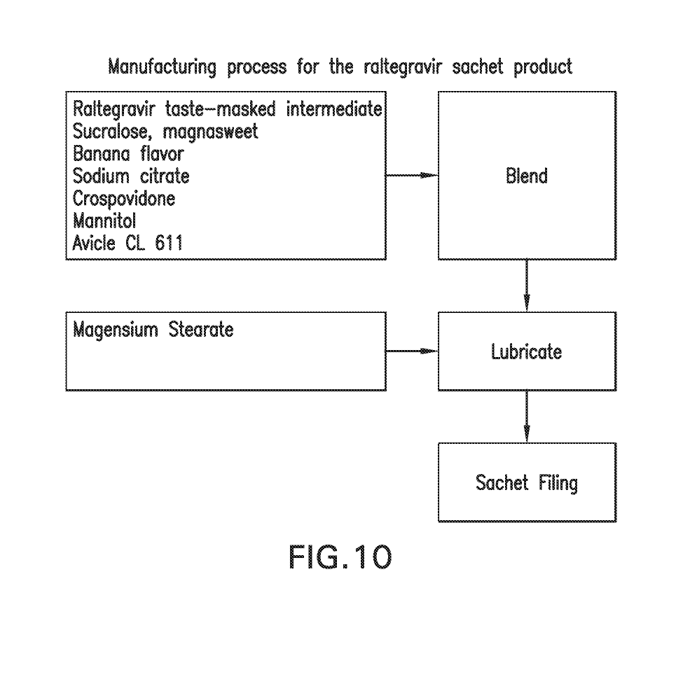

The raltegravir coated granules for incorporation into the sachet are identical to those in the chewable tablet. The formulation is adjusted somewhat to accommodate the fact that the sachet granules are intended to be dosed as an aqueous suspension. FIG. 10 is a block flow diagram of the process used to generate the raltegravir sachet product. The extragranular excipients are added as screened powders in a blending step with the coated granules prior to sachet filling. The gross composition of the sachet is displayed in Table 7.

TABLE 7

| Ingredient | Function | Composition per sachet |
|---|---|---|
| Raltegravir | Active | 108.72 |
| Hydroxypropyl cellulose | Binder | 9.33 |
| Ethycellulose | Coating | 10.73 |
| Opadry I Clear | Coating | 6.999 |
| Crospovidone | Disintegrant | 23.33 |
| Mannitol | Diluent | 193.17 |
| Sucralose | Sweetener | 2.333 |
| MagnaSweet | Sweetener | 2.333 |
| Banana Flavor | Flavor | 9.332 |
| Avicel CL-611 | Suspending agent | 93.32 |
| Magnesium Stearate | Lubricant | 6.999 |

FIG. 11 displays the effect of product temperature on the particle size distribution of uncoated granules for several process conditions at the Niro MP2 30 L scale. The data show that as temperature rises, the particle size distribution shifts to the left (i.e., particles get smaller) and the distribution flattens somewhat (i.e., there is a greater variety of particle sizes present).

### Pharmacokinetics

The relative bioavailability of a raltegravir pediatric ethylcellulose formulation and the raltegravir adult tablet formulation in healthy adults was evaluated in a clinical study known as P031. P031 was an open-label, randomized, single-dose, 2-period crossover study in healthy adult subjects. Twelve (12) healthy adult male and female subjects received 2 treatments (Treatments A and B) in a randomized crossover manner Treatment A consisted of a 100-mg single oral dose of the raltegravir adult tablet formulation. Treatment B consisted of a 100-mg single oral dose of the initial raltegravir pediatric ethylcellulose tablet formulation. All doses were administered in the fasted state. There was a minimum of a 4-day postdose washout interval between treatment periods.

In comparison of the pharmacokinetic profiles achieved following administration of the raltegravir pediatric ethylcellulose tablet formulation and the raltegravir adult poloxamer tablet formulation in healthy adult subjects, the geometric means for C₁₂ₕᵣ were similar, while AUC(0-∞) and Cₘₐₓ values for the pediatric formulation were higher than their corresponding geometric means for the adult poloxamer formulation (see Table 8). The median Tₘₐₓ value for the pediatric formulation was modestly shorter relative to the adult poloxamer formulation. Half-life values for both the initial (α) and terminal (β) phases were similar for both formulations. The results were consistent with there being some difference in the absorption portion of the pharmacokinetic profile between the formulations, but little difference in the later part of the profile.

The target population of the pediatric formulation is young children, a population in which palatability issues are of concern. A subjective evaluation of taste of the pediatric tablet formulation was conducted in this study and revealed that there were overall no major palatability concerns.

Table 8 displays comparative data collected in study P031 regarding raltegravir plasma pharmacokinetics following administration of single 100-mg oral doses of the raltegravir adult poloxamer and pediatric ethylcellulose tablet formulations to healthy, adult, male and female subjects.

TABLE 8

| | | Pediatric | | Poloxamer | | Pediatric/ | |
|---|---|---|---|---|---|---|---|
| Pharmacokinetic | | Formulation | | Formulation | | Poloxamer | |
| Parameter | N | GM^{†} | 95% CI | GM^{†} | 95% CI | GMR | 90% CI |
| C_{12 hr} (nM) | 12 | 31.9 | (22.7, 44.9) | 29.5 | (21.0, 41.5) | 1.08 | (0.83, 1.42) |
| AUC(0-∞) | 12 | 7.96 | (6.59, 9.63) | 4.31 | (3.56, 5.21) | 1.85 | (1.46, 2.34) |
| (μM · hr) | | | | | | | |
| Cₘₐₓ (μM) | 12 | 3.39 | (2.67, 4.30) | 1.18 | (0.93, 1.50) | 2.87 | (2.18, 3.80) |
| Tₘₐₓ (hr) | 12 | 1.0^{‡} | (0.5, 1.5)^{‡} | 2.5^{‡} | (0.5, 4.0)^{‡} | −1.75^{∥} | (−2.25, −1.00)^{∥} |
| t_{1/2} α (hr) | 12 | 0.90^{§} | (0.30)^{§} | 0.97^{§} | (0.30)^{§} | −0.07^{∥} | (−0.35, 0.20)^{∥} |
| t_{1/2} β (hr) | 12 | 4.7^{§} | (1.7)^{§} | 5.2^{§} | (1.7)^{§} | −0.35^{∥} | (−1.50, 5.15)^{∥} |

^{†}Geometric mean computed from least squares estimate from the mixed model performed on the natural-log transformed.
^{‡}Median (minimum, maximum) provided for Tₘₐₓ values.
^{§}Harmonic mean (pseudo SD) provided for t_{1/2}.
^{∥}Median difference (pediatric − poloxamer) and CI from Hodges-Lehmann estimation reported for Tₘₐₓ and t_{1/2}.
GMR: Geometric mean ratio, CI: confidence interval.

The relative bioavailability of a raltegravir pediatric ethylcellulose chewable tablet formulation, a raltegravir oral granules for suspension formulation, and the raltegravir adult poloxamer tablet formulation in healthy adults was evaluated in a study known as P068. P068 was an open label, 4-period, randomized, crossover study in healthy, adult, male and female subjects. Twelve (12) subjects each received 4 treatments (Treatments A, B, C, and D) randomized in a balanced, crossover design in Periods 1 through 4. Treatment A consisted of a single oral dose of 400 mg raltegravir adult formulation tablet. Treatment B consisted of a single oral dose of 400 mg raltegravir ethylcellulose pediatric chewable tablet formulation. Treatment C consisted of a single oral dose of 400 mg raltegravir oral granules in a liquid suspension. Treatment D consisted of a single oral dose of 400 mg raltegravir ethylcellulose pediatric chewable tablet formulation administered following a high fat meal. Treatments A-C were administered in the fasted state. There was a minimum of 4 days of washout between the single doses in each treatment period.

The geometric mean pharmacokinetic parameter values for the raltegravir pediatric chewable tablet and oral granules for suspension formulations were estimated and compared to the corresponding values for the adult tablet formulation, all following single dose administration of 400 mg in the fasted state.

The geometric mean C₁₂ₕᵣ values were similar for all formulations, while AUC(0-∞) and Cₘₐₓ values were higher for both the pediatric chewable tablet formulation and the oral granules formulation compared to the adult tablet formulation. For the oral granules formulation, AUC(0-∞) and Cₘₐₓ were moderately higher (2.6- and 4.6-fold) than those obtained with the adult tablet formulation and slightly higher (1.5- and 1.4-fold) than those obtained with the pediatric chewable tablet formulation. Both the pediatric chewable tablet formulation and oral granules formulations had earlier median Tₘₐₓ values compared with the adult tablet formulation (0.5 and 1.0 hours for the chewable tablet and oral granules formulations, respectively, compared to 4.0 hours for the adult tablet formulation). Half-life values for both the initial (α) and terminal (β) phases were similar for all formulations. The results were consistent with there being some difference in the absorption portion of the pharmacokinetic profile among the formulations, but little difference in the later part of the profile.

The higher AUC(0-∞) and Cₘₐₓ values for the oral granules formulation are not expected to have any meaningful clinical consequences. The pharmacokinetic properties of the two pediatric formulations are similar to the raltegravir Phase I lactose formulation, which was well-tolerated in adults, as shown in study P007. Based on the absence of a statistically significant difference in trough values, and the otherwise moderate dissimilarities in the other pharmacokinetic parameters, these results supported continued clinical development of both pediatric formulations.

Table 9 displays comparative data regarding raltegravir plasma pharmacokinetics following single-dose administration of the raltegravir adult tablet formulation, ethylcellulose pediatric chewable tablet formulation (fasted or fed), and raltegravir oral granules in a liquid suspension collected in study P068.

TABLE 9

| Pharmacokinetic | | A^{†} | B^{†} | C^{†} | D^{†} | | |
|---|---|---|---|---|---|---|---|
| Parameter (Units) | N | GM | GM | GM | GM | Comparison | GMR (90% CI) |
| C_{12 hr} (nM)^{§} | 12 | 149 | 134 | 162 | 387 | Treatment C/Treatment A | 1.09 (0.84, 1.41) |
| | | | | | | Treatment C/Treatment B | 1.20 (0.92, 1.56) |
| | | | | | | Treatment D/Treatment B | 2.88 (2.21, 3.75) |
| | | | | | | Treatment B/Treatment A | 0.90 (0.70, 1.18) |
| AUC_{0-∞} (μM · hr)^{§} | 12 | 19.2 | 34.2 | 50.4 | 32.3 | Treatment C/Treatment A | 2.62 (2.17, 3.17) |
| | | | | | | Treatment C/Treatment B | 1.47 (1.22, 1.78) |
| | | | | | | Treatment D/Treatment B | 0.94 (0.78, 1.14) |
| | | | | | | Treatment B/Treatment A | 1.78 (1.47, 2.15) |
| Cₘₐₓ (μM)^{§} | 12 | 5.00 | 16.1 | 23.2 | 6.14 | Treatment C/Treatment A | 4.64 (3.41, 6.30) |
| | | | | | | Treatment C/Treatment B | 1.44 (1.06, 1.95) |
| | | | | | | Treatment D/Treatment B | 0.38 (0.28, 0.52) |
| | | | | | | Treatment B/Treatment A | 3.22 (2.37, 4.38) |
| Tₘₐₓ (hr) | 12 | 4.0 | 0.5 | 1.0 | 1.0 | | |
| t_{1/2I} (hr)^{¶} | | 1.5 (0.3) | 1.7 (0.2) | 1.6 (0.3) | 2.0 (0.6) | | |
| t_{1/2T} (hr)^{¶} | 12 | 9.0 (5.9) | 9.3 (5.1) | 10.0 (3.2) | 9.2 (3.8) | | |

^{†}Treatment A = 400 mg Raltegravir, poloxamer (administered fasted).
Treatment B = 400 mg Raltegravir, EC (administered fasted).
Treatment C = 400 mg Raltegravir, OG in a liquid suspension (administered fasted).
Treatment D = 400 mg Raltegravir, EC (administered with a high-fat meal).
^{§}Back-transformed least squares mean and confidence interval from mixed effects model performed on the natural log-transformed values.
Median values presented for Tₘₐₓ.
^{¶}Harmonic mean (jack-knife standard deviation) values presented for t_{1/2I} and t_{1/2T}. For t_{1/2t}, the N's for Treatments A, B, C, and D are 11, 12, 12, and 10, respectively.

As described above, the effect of a high-fat meal on the pharmacokinetic profile of the pediatric chewable tablet was assessed in P068. A similarly designed food effect study (comparing single dose administration with a high-fat meal vs. fasted) was previously conducted for the adult tablet formulation. In this study, the raltegravir AUC(0-∞) geometric mean ratio (fed/fasted) and corresponding 90% CI were 1.19 μm·hr (0.91 μm.hr, 1.54 μm.hr). The geometric mean ratio (fed/fasted) and 90% confidence interval for Cₘₐₓ and C₁₂ₕᵣ were 0.66 μm (0.44 μm, 0.98 μm) and 8.5 μm (5.5 μm, 13.1 μm), respectively, while the median and 90% CI for the difference between Tₘₐₓ in the fed and fasted states was 7.3 hr (5.8 hr, 8.8 hr). The decreased Cₘₐₓ and similar AUC(0-∞) values observed for both formulations following administration with a high fat meal provide the grounds for the inference that a high fat meal slows the rate of absorption of raltegravir from both formulations, with no meaningful change in extent of absorption. The effect of a high-fat meal on both formulations is qualitatively similar, though a longer delay in Tₘₐₓ was observed for the adult formulation compared to the pediatric formulation (median difference of 7.3 hours for the adult formulation versus 0.5 hours for the pediatric formulation), and a correspondingly larger increase in C₁₂ₕᵣ was observed for the adult formulation (mean 8.5-fold increase for the adult formulation versus 2.9-fold increase for the pediatric formulation). For both formulations, administration with food increases variability in the pharmacokinetic parameter values.

As described above, the pharmacokinetics of several potential pediatric formulations were assessed in biocomparison studies in a healthy adult population. All of the potential tablet formulations were designed to dissolve or be chewed in the mouth before swallowing, and so would be expected to release drug more quickly than the currently approved adult formulation, which was designed to be an erodible tablet. It is therefore not surprising that all of the candidate pediatric tablet formulations had somewhat higher AUC(0-∞) and Cₘₐₓ values compared to the adult formulation. In particular, the pediatric chewable tablet formulation chosen for use in study P1066 had an average 1.8-fold higher AUC(0-∞) and 3.2-fold higher Cₘₐₓ compared to the adult formulation. Peak concentrations also occurred earlier for the pediatric chewable tablet formulation (in the fasted state, median Tₘₐₓ=0.5 hr for the pediatric chewable tablet versus 4 hr for the adult formulation). C₁₂ₕᵣ concentrations and half-lives were similar for both formulations, though, from which it may be inferred that the differences in pharmacokinetic profile are largely attributable to differences in absorption.

The somewhat higher AUC(0-∞) and Cₘₐₓ for the pediatric formulation are not expected to have any meaningful clinical significance. The pharmacokinetic properties of the pediatric formulation are similar to the raltegravir Phase I lactose formulation, which was well-tolerated in adults. In a comparison of the Phase I lactose formulation to the Phase I poloxamer formulation, which has a similar pharmacokinetic profile to the final market composition poloxamer formulation, raltegravir Phase I lactose AUC(0-∞) and Cₘₐₓ were ˜1.6-fold and ˜2.4-fold that of the respective values of the poloxamer formulation, as shown in study P007. To date, no acute safety findings have been identified that were temporally associated with peak concentrations, and raltegravir has been found to be generally well-tolerated in the clinical program with no dose-related toxicities. Effects of up to a 2-fold increase in AUC in the adult development program were considered to be not clinically relevant based on available clinical experience with regard to safety. The pediatric chewable tablet studied in P068 has the same composition as the formulation used in study P1066, which led to an acceptable pharmacokinetic, efficacy, and safety profile in pediatric patients. Alternate formulations, dosage strengths, and dosing regimens that are bioequivalent to those explicitly described herein are within the scope of the invention.

Raltegravir has been studied in 126 antiretroviral treatment-experienced HIV-1 infected pediatric patients 2 through 18 years of age, in combination with other antiretroviral agents in IMPAACT P1066. Of the 126 patients, 96 received only the final recommended dose through at least 24 weeks.

Frequency, type and severity of drug related adverse reactions in pediatric patients were comparable to those observed in adults. Grade 3 or higher adverse reactions were reported in one patient (psychomotor hyperactivity, abnormal behavior, insomnia) and one serious Grade 2 possibly drug related allergic rash was reported.

Grade 3 or 4 laboratory abnormalities were neutrophil count decreased (Grade 3: 7.4%; Grade 4: 1.1%), GGT increased (Grade 3: 1.1%), magnesium decreased (Grade 3: 1.1%), glucose decreased (Grade 3: 1.1%), glucose abnormal (Grade 3: 1.1%); creatinine increased (Grade 3: 1.1%), bilirubin increased (Grade 3: 1.1%), ALT increased (Grade 3: 1.1%), AST increased (Grade 3: 1.1%; Grade 4: 1.1%).

### Dosing

Based on the pharmacokinetic data, weight-based dosing scheme of 6 mg/kg twice daily (BID) would be implemented. Thus, for the treatment of pediatric patients with HIV-1 infection, the dosage of raltegravir was fixed as follows:

12 years of age and older: One 400 mg tablet administered orally, twice daily

6 through 11 years of age (2 dosing options):
One 400 mg tablet administered orally, twice daily (if at least 25 kg in weight) or
Chewable tablets: weight based dosing to maximum dose 300 mg, administered twice daily as specified in Table 10.

2 through 5 years of age:
Chewable tablets: weight based dosing to maximum dose 300 mg, administered twice daily as specified in Table 10.

Displays recommended doses for raltegravir chewable tablets in pediatric patients who are 2 through 11 years of age.

TABLE 10

Patient Body Weight

| (kg) | (lbs) | Dose |
|---|---|---|
| 7 through 10 | 15 through 22 | 50 mg twice daily |
| 10 through 14 | 22 through 31 | 75 mg twice daily |
| 14 through 20 | 31 through 44 | 100 mg twice daily |
| 20 through 28 | 44 through 62 | 150 mg twice daily |
| 28 through 40 | 62 through 88 | 200 mg twice daily |
| at least 40 | at least 88 | 300 mg twice daily |

The maximum dose of chewable tablets is 300 mg twice daily.

### Therapeutic Uses

The methods of the present invention involve the use of compounds of the present invention in the inhibition of HIV protease (e.g., wild type HIV-1 and/or mutant strains thereof), the prophylaxis or treatment of infection by human immunodeficiency virus (HIV) and the prophylaxis, treatment or delay in the onset or progression of consequent pathological conditions such as AIDS. Prophylaxis of AIDS, treating AIDS, delaying the onset or progression of AIDS, or treating or prophylaxis of infection by HIV is defined as including, but not limited to, treatment of a wide range of states of HIV infection: AIDS, ARC (AIDS related complex), both symptomatic and asymptomatic, and actual or potential exposure to HIV. For example, the present invention can be employed to treat infection by HIV after suspected past exposure to HIV by such means as blood transfusion, exchange of body fluids, bites, accidental needle stick, or exposure to patient blood during surgery.

As noted above, the present invention is also directed to use of a formulation of raltegravir with one or more anti-HIV agents. An “anti-HIV agent” is any agent that inhibits HIV replication or infection, the treatment or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. For example, the compounds of this invention may be effectively administered, whether at periods of pre-exposure and/or post-exposure, in combination with effective amounts of one or more anti-HIV agents selected from HIV antiviral agents, antiinfectives, or vaccines useful for treating HIV infection or AIDS. Suitable HIV antivirals for use in combination with the compounds of the present invention include, for example, those listed in Table 11 as follows:

TABLE 11
Antiviral Agents for Treating HIV infection or AIDS

| Name | Type |
|---|---|
| abacavir, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, azidothymidine, Retrovir ® | nRTI |
| Capravirine | nnRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, DLV, Rescriptor ® | nnRTI |
| efavirenz, EFV, Sustiva ®, Stocrin ® | nnRTI |
| efavirenz + emtricitabine + tenofovir DF, Atripla ® | nnRTI + nRTI |
| emtricitabine, FTC, Emtriva ® | nRTI |
| emtricitabine + tenofovir DF, Truvada ® | nRTI |
| emvirine, Coactinon ® | nnRTI |
| enfuvirtide, Fuzeon ® | FI |
| enteric coated didanosine, Videx EC ® | nRTI |
| etravirine, TMC-125 | nnRTI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| Lopinavir | PI |
| lopinavir + ritonavir, Kaletra ® | PI |
| maraviroc, Selzentry ® | EI |
| nelfinavir, Viracept ® | PI |
| nevirapine, NVP, Viramune ® | nnRTI |
| PPL-100 (also known as PL-462) (Ambrilia) | PI |
| ritonavir, Norvir ® | PI |
| saquinavir, Invirase ®, Fortovase ® | PI |
| stavudine, d4T, didehydrodeoxythymidine, Zerit ® | nRTI |
| tenofovir DF (DF = disoproxil fumarate), TDF, | nRTI |
| Viread ® | |
| tipranavir, Aptivus ® | PI |

EI = entry inhibitor;
FI = fusion inhibitor;
InI = integrase inhibitor;
PI = protease inhibitor;
nRTI = nucleoside reverse transcriptase inhibitor;
nnRTI = non-nucleoside reverse transcriptase inhibitor.
Some of the drugs listed in the table are used in a salt form; e.g., abacavir sulfate, indinavir sulfate, atazanavir sulfate, nelfinavir mesylate.

It is understood that the scope of combinations of the formulations of this invention with anti-HIV agents is not limited to the HIV antivirals listed in Table 10 and/or listed in the above-referenced Tables in WO01/38332 and WO02/30930, but includes in principle any combination with any pharmaceutical composition useful for the treatment or prophylaxis of AIDS. The HIV antiviral agents and other agents will typically be employed in these combinations in their conventional dosage ranges and regimens as reported in the art, including, for example, the dosages described in the Physicians' Desk Reference, Thomson PDR, Thomson PDR, 57^{th} edition (2003), the 58^{th} edition (2004), or the 59^{th} edition (2005). The dosage ranges for a compound of the invention in these combinations are the same as those set forth above.

Other than as shown in the operating example or as otherwise indicated, all numbers used in the specification and claims expressing quantities of ingredients, reaction conditions, and so forth, can be modified in all instances by the term “about.” The above description is not intended to detail all modifications and variations of the invention. It will be appreciated by those skilled in the art that changes can be made to the embodiments described above without departing from the inventive concept. It is understood, therefore, that the invention is not limited to the particular embodiments described above, but is intended to cover modifications that are within the spirit and scope of the invention, as defined by the language of the following claims. While the foregoing specification teaches the principles of the present invention, with examples provided for the purpose of illustration, the practice of the invention encompasses all of the usual variations, adaptations and/or modifications that come within the scope of the following claims. All publications, patents and patent applications cited herein are incorporated by reference in their entirety into the disclosure.

1. A pharmaceutical composition for oral administration wherein the active agent consists of raltegravir potassium and which comprises a plurality of taste-masked granules, each of which comprises:
(i) a core granule comprising raltegravir potassium and a binder, and
(ii) a taste-masking polymer composition that forms a coating on the core granule, wherein said taste-masking polymer composition comprises an ethyl cellulose dispersion, wherein said pharmaceutical composition is in the form of a chewable tablet or sachet.

2. (canceled)

3. (canceled)

4. The composition according to claim 1, wherein said raltegravir potassium is anhydrous, and in crystalline Form 1, which is characterized by an X-ray powder diffraction pattern obtained using copper K_{α} radiation which comprises 2θ values in degrees of about 5.9, about 12.5, about 20.0, about 20.6 and about 25.6.

5. (canceled)

6. The composition according to claim 1, wherein said raltegravir potassium is anhydrous, and in crystalline Form 1, which is characterized by a differential scanning calorimetry curve, obtained at a heating rate of 10° C./min in a closed cup under nitrogen, exhibiting a single endotherm with a peak temperature of about 279° C.

7.-8. (canceled)

9. The composition according to claim 1, wherein said raltegravir potassium is anhydrous, and in crystalline Form 2, which is characterized by an X-ray powder diffraction pattern obtained using copper K_{α} radiation which comprises 2θ values in degrees of about 7.9, about 13.8, about 15.7, about 24.5 and about 31.5.

10.-13. (canceled)

14. The composition according to claim 1, wherein said raltegravir potassium is anhydrous, and in crystalline Form 3, which is characterized by an X-ray powder diffraction pattern obtained using copper K_{α} radiation which comprises 2θ values in degrees of about 7.4, about 7.8, about 12.3, about 21.6 and about 24.7.

15.-17. (canceled)

18. The composition according to claim 1, wherein said binder is hydroxypropyl cellulose.

19. The composition according to claim 1, wherein the weight ratio of said binder to said raltegravir potassium is between about 0.06 and about 0.08.

20. (canceled)

21. The composition according to claim 1, wherein the amount of raltegravir in the composition is about 25 mg, about 50 mg, or about 100 mg, or the salt-equivalent thereof.

22. The composition according to claim 1, further comprising an extragranular matrix, said matrix comprising at least one flavoring agent, a disintegrant, a filler and a lubricant.

23. The composition according to claim 1, wherein said composition is a tablet or capsule.

24. A composition according to claim 1 selected from Compositions A, B and C, wherein said compositions are comprised of the following:

| | Composition | | |
|---|---|---|---|
| | A (mg) | B (mg) | C (mg) |
| Raltegravir | 27 | 54 | 109 |
| Hydroxypropyl | 2 | 5 | 9 |
| cellulose | | | |
| Ethyl cellulose | 3 | 5 | 11 |
| dispersion | | | |
| Film coating blend, | 2 | 4 | 7 |
| clear Opadry | | | |
| Total Coated | 34 | 68 | 136 |
| Raltegravir Granule | | | |
| Extragranular | | | |
| Saccharin sodium | 7 | 7 | 14 |
| Sucralose | 2 | 2 | 5 |
| Banana Flavor | 5 | 5 | 9 |
| Orange Flavor | 7 | 7 | 14 |
| Monoammonium | 2 | 2 | 5 |
| Glycyrrhizinate | | | |
| Flavor Masking | 5 | 5 | 9 |
| Neutral | | | |
| Sodium Citrate | 1 | 1 | 2 |
| Dihydrate | | | |
| Mannitol | 154 | 120 | 240 |
| Crospovidone | 12 | 12 | 23 |
| Ferric Oxide, yellow | 1 | 1 | 2 |
| Sodium Stearyl | 2 | 2 | 5 |
| Fumarate | | | |
| Magnesium Stearate | 1 | 1 | 2 |

25. The composition according to claim 1, wherein said composition is a sachet for aqueous suspension prior to administration, further comprising at least one flavoring agent, a disintegrant, a suspending agent, a lubricant and a diluent.

26. The composition according to claim 25 comprising the following:

| | Composition per sachet |
|---|---|
| Ingredient | (mg) |
| Raltegravir | 109 |
| Hydroxypropyl cellulose | 9 |
| Ethycellulose | 11 |
| Opadry I Clear | 7 |
| Crospovidone | 23 |
| Mannitol | 194 |
| Sucralose | 2 |
| MagnaSweet | 2 |
| Banana Flavor | 9 |
| Avicel CL-611 | 9 |
| Magnesium Stearate | 7 |

27. The pharmaceutical composition according to claim 1, wherein said core granules have a D₅₀ of between about 130 and about 330 μm.

28. (canceled)

29. The pharmaceutical composition according to claim 1, wherein said coated granules have a D₅₀ of between about 180 and about 340 μm.

30. (canceled)

31. The pharmaceutical composition according to claim 1, wherein administration of said composition to a person results in a mean blood plasma concentration of raltegravir displaying a Cₘₐₓ of about 3.4 μm and an AUC of about 8 μm·hr.

32. (canceled)

33. A method of treating HIV in a person in need of such treatment comprising to said person a pharmaceutical composition according to claim 1, wherein said dose of raltegravir is determined as follows:
Patient Body Weight

| (kg) | (lbs) | Dose |
|---|---|---|
| 7 through 10 | 15 through 22 | 50 mg twice daily |
| 10 through 14 | 22 through 31 | 75 mg twice daily |
| 14 through 20 | 31 through 44 | 100 mg twice daily |
| 20 through 28 | 44 through 62 | 150 mg twice daily |
| 28 through 40 | 62 through 88 | 200 mg twice daily |
| at least 40 | at least 88 | 300 mg twice daily |

34. (canceled)

35. (canceled)

36. A process for preparing a pharmaceutical composition comprising the steps of:
a) granulating raltegravir and a binder into granules by a Wurster granulating process;
b) coating said granules with a taste-masking agent by a Wurster coating process;
c) preparing an extragranular matrix comprising a binder, a disintegrant, a flavoring agent and a lubricant; and,
d) compressing said granules and said matrix into a tablet.

37. (canceled)

38. The composition according to claim 1 comprising 27.16 mg of raltegravir potassium, hydroxypropyl cellulose, an ethyl cellulose dispersion, a film coating blend, saccharin sodium, sucralose, banana flavor, orange flavor, monoammonium glycyrrhizinate, flavor masking neutral, sodium citrate dehydrate, mannitol, crospovidone, ferric oxide yellow, ferric oxide red, sodium stearyl fumarate and magnesium stearate.

39. The composition according to claim 1 comprising 108.6 mg of raltegravir potassium, hydroxypropyl cellulose, an ethyl cellulose dispersion, a film coating blend, saccharin sodium, sucralose, banana flavor, orange flavor, monoammonium glycyrrhizinate, flavor masking neutral, sodium citrate dehydrate, mannitol, crospovidone, ferric oxide yellow, ferric oxide red, sodium stearyl fumarate and magnesium stearate.
